(19) United States
(12) Patent Application Publication
(10) Pub. No.: US 2023/0172589 A1
(43) Pub. Date: Jun. 8, 2023

(54) A SWAB

(71) Applicant: NATIONAL UNIVERSITY OF SINGAPORE, Singapore (SG)

(72) Inventors: Ching Chiuan YEN, Singapore (SG); Wai Tung CHOW, Singapore (SG); Chee Keong CHEN, Singapore (SG); Willie TAY, Singapore (SG)

(21) Appl. No.: 17/922,909

(22) PCT Filed: Apr. 30, 2021
(86) PCT No.: PCT/SG2021/050239

§ 371 Date: Nov. 2, 2022

(30) Foreign Application Priority Data
May 6, 2020 (SG) 10202004195S
Jun. 2, 2020 (SG) 10202005209W

(51) Int. Cl. A61B 10/00; B01L 3/00

(52) U.S. Cl. CPC A61B 10/0051; B01L 3/5029; B01L 2300/161; B01L 2300/047

(57) ABSTRACT

Described is a tip for a swab for collecting a specimen from a subject, the tip being shaped for insertion into, and collection of the specimen from, a body cavity of the subject such as the nasal cavity. The tip comprises a plurality of curved blades defining an internal reservoir for receiving the specimen, the tip being attached or attachable to a rod. In the preferred embodiment, each of the blade comprises helical turns.

### TECHNICAL FIELD

The present invention relates to a swab for collecting a sample from a body cavity, such as a nasopharyngeal swab.

### BACKGROUND

Devices, such as swabs, for collecting biological specimens of organic material are known in the field of clinical and diagnostic analyses. These swabs generally include a cylindrical rod on which an end or tip is formed from a wad of fiber material, such as rayon or a natural fibre such as cotton. The tip has hydrophilic properties to allow rapid absorption of the specimen from a body cavity. Stable adherence of the fibre wrapped around the end or tip of the rod or stick is generally achieved by gluing.

Collection swabs containing the collected material are often immersed in a viral transport medium in a receptacle such as a test tube, vial, culture dish, or culture bottle, soon or immediately after collection. This is to preserve and conserve the collected specimen during storage and/or transport to, for example, a laboratory.

Given the essentially random arrangement of fibres in the tip, the fibres may block part of the sample from release into the viral transport medium. The tip may &so entrain air which can further inhibit release, of the sample. As a result, the amount of the sample released into the viral transport medium is unpredictable.

In addition, flocked swab tips and other designs can cause discomfort due to the angle at which some fibres scrape the sample from the nasal cavity or other orifice. Also, the small fibre diameter can be an irritant in sensitive cavities such as nostrils, particularly where the sample is being taken from a patient whose sinuses are already sensitive due to illness. Moreover, the randomness of the arrangement of fibres may result in a large number of rotations of the swab being necessary for a user to be confident that a viable sample volume has been collected.

Some swabs have been proposed that include tip having a well-defined structure or web to facilitate release of the sample. That structure is susceptible to plastic deformation that reduces the contact between the tip and internal wall of the body cavity. In some cases a portion of the tip can break away, potentially injuring the subject from whom the sample is being taken. Such plastic deformation can occur as a result of the swab being grasped during withdrawal from packaging or otherwise during use.

It would be desirable to overcome or ameliorate at least one of the above-described problems, or at least to provide a useful alternative.

### SUMMARY

Described herein is a tip for a swab for collecting a specimen from a subject. The tip is shaped for insertion into, and collection of the specimen from, a body cavity of the subject. The tip comprises a plurality of curved blades defining an internal reservoir for receiving the specimen, and is attached or attachable to a rod. The user grasps and manipulates the rod to collect a sample in or on the tip, from the subject.

The tip has a length and each blade may be continuous along the length.

Each curved blade may be a helical blade. Each curved blade may comprise between one and two helical turns. Each curved blade may comprise about 1.5 helical turns. The helical turns occur about a longitudinal axis of the tip, and thus also of the rod from which the tip extends or to which the tip is attached in use.

The curved blades may be equidistantly, circumferentially spaced around the tip.

The tip may comprise between five and nine curved blades.

The curved blades may have an outer side for contacting an internal wall of the cavity. The curved blades may each also have the inner side with the internal reservoir being defined between the inner sides. The curved blades may be spaced to transfer the specimen from the cavity to the reservoir under capillary action. The spacing between blades may work with the surface tension, or expected surface tension, of the sample such that the surface tension draws the sample between the blades and into the cavity—i.e. capillary action.

Each curved blade may extend along a coterminous length of the tip. That length may be the full length of the tip.

The curved blades may curve in a common direction. The tip, or the plurality of blades, may further comprise a plurality of oppositely curved blades. The oppositely curved blades may curve in a direction opposite to the common direction. The oppositely curved blades may be offset inwardly of the curved blades. In this sense, the curved blades and oppositely curved blades may form concentric circles when the tip is viewed in cross-section, with the curved blades surrounding the oppositely curved blades. Each oppositely curved blade may be a helical blade. The oppositely curved blades may have an outer side for contacting (i.e. that contacts) the curved blades. The internal reservoir may also be defined between an inner sides of the oppositely curved blades.

The tip may further comprise one or more perpendicular supports for supporting the blades between opposite ends of the tip.

Each blade may comprise a square, flat diamond-shaped or diamond-shaped cross-section.

A surface of the curved blades may be textured.

The curved blades may each form a helix and curve in a first direction. The tip, or the curved blades, may further comprise a plurality of oppositely curved blades that curve in a second direction opposite the first direction. The oppositely curved blades may be offset inwardly of the curved blades. The curved blades and oppositely curved blades may define diamond-shaped apertures through which the sample is conveyed into the reservoir. The diamond-shaped apertures may be longer in a longitudinal direction of the tip than in a transverse direction.

The blades may have sufficient rigidity to scrape a sample from an internal wall of the cavity. The blades may be of sufficient flexibility to vary a spacing between neighbouring ones of said blades, during rotation of the tip in the cavity, to drive the sample into the reservoir.

Also disclosed herein is a swab comprising:
a tip as described above; and
a rod having a distal end, the tip being located at the distal end.

The rod may be integrally formed with the tip.

The rod may be attached to the tip. The attachment may be by friction fit, threaded fit—e.g. the tip may have one of the male and female screw thread and the rod may have the other of the male and female screw thread.

Advantageously, in some embodiments the shape of the blades ensures a smooth movement of the blades against the internal wall of the body cavity from which the sample is being collected, reducing irritation when compared with flocked or cotton swabs.

Advantageously, in some embodiments the shape of the blades causes the sample to be drawn radially inwardly to the reservoir, in which it is retained until the tip is deposited in a viral transport medium.

Advantageously, due to the regular, open structure of the tip, viral transport medium can readily access the internal reservoir to release the sample retained therein.

### BRIEF DESCRIPTION OF THE DRAWINGS

Embodiments of the present invention will now be described, by way of non-limiting example, with reference to the drawings in which:

FIG. 1 is a side view of a swab in accordance with present teachings;

FIG. 2 is a close-up view of the tip of the swab of FIG. 1;

FIG. 3, comprising images (a) to (d), shows various embodiments of a swab in accordance with present teachings, having different tip dimensions and number of blades in the tip;

FIG. 4, comprising images (a) to (f), shows various components and features of an alternative embodiment of a tip of a swab in accordance with present teachings;

FIG. 5, comprising images (a) to (d), shows embodiments of a swab, illustrating variation in dimensions of the tip, the number of blades in the tip and tips with and without a set of oppositely curved blades within the set of curved blades; and

FIG. 6, comprising images (a) to (c), illustrates a further embodiment of a swab in accordance with present teachings.

### DETAILED DESCRIPTION

The present tips and swabs incorporating those tips may be used as nasopharyngeal swabs. Clinical requirements provide that nasopharyngeal (NP) swabs need to be able to capture cells and mucus (i.e. the sample), retain cells and mucus (the sample should not fall off the tip before getting into transport media) and the release of cells and mucus in the transport media (which will allow RNA to be measured/virus to be detected).

Tips disclosed herein have a regular (i.e. repeating and predetermined) structure generally comprising curved blades extending along the tip. The blades define an internal reservoir in which a sample is collected from a subject. The curvature of the blades ensure that, at any particular time during sample collection, multiple blades are in contact with the internal wall of the body cavity from which the sample is being collected. This assists in distributing the pressure applied to the internal wall, along the length of the tip. The result is that greater sample volume can be obtained while causing less discomfort to the subject, when compared with some designs such as flocked swabs. In addition, the blades may serve as a form of Archimedes' Screw, by drawing sample towards the opening via which the swab was inserted into the body cavity.

For ease of illustration, the tips disclosed herein will be depicted attached to a rod to form a complete swab. It will be appreciated, however, that particular embodiments may allow for a tip to be produced separately from the rod and attached thereto.

Particular embodiments of the present invention have a structure that is designed to resist plastic deformation under lateral compression applied at the tip. The structure is also designed to release the sample—e.g. under surface tension in the reservoir in the tip—while facilitating ready release upon immersion of the tip in a viral transport medium (VTM).

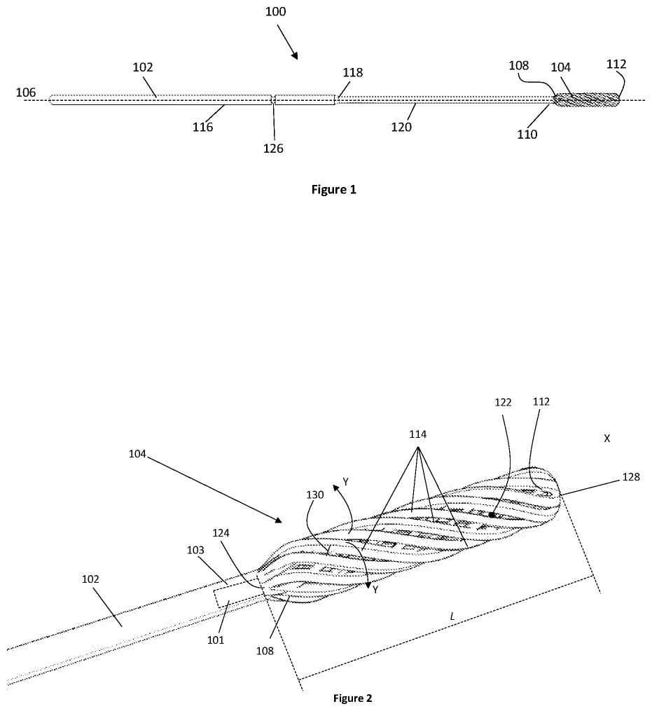

Such a tip 104 is shown in FIG. 1, with rod 102, the tip 104 and rod 102 together forming a swab 100. The swab 100 is used for collecting a specimen from a subject. In the present embodiment, the rod 102 and tip 104 are integrally formed—e.g. by 3-dimensional printing. The rod 102 and tip 104 may instead be separately formed, with the rod subsequently attached thereto. Such an arrangement is illustrated in broken lines in FIG. 2, in which a male member 101 of the tip 104 is received in a female member 103 of the rod 102, and attaches thereto—e.g. by cooperating screw threads, adhesion, friction or other attachment mechanism. In such embodiments, the tip 104 is assembled onto the rod 102 to form the swab 100, prior to use.

The rod 102 has a longitudinal axis 106. During use, the swab 100 is inserted into a body cavity of a subject and the rod 102 is rotated about the longitudinal axis 106 to collect a sample from the internal wall of the body cavity. The tip 104 has a proximal region 108 at a distal end 110 of the rod 102, and a distal region 112 opposite the proximal region 108.

In general, the rod 102 will be formed in a cylindrical shape. The longitudinal axis 106 passes through the centre of the cylinder. The rod 102 comprises a handling section 116 and a tapered section 118. The tapered section 118 extends distally of the handling section 116. The tapered section 118 extends from the larger diameter handling section 116 to a smaller diameter end section 120 that includes the distal end of the rod 102. In other embodiments, the rod may include a further tapered section extending from the smaller diameter section 120 to yet a narrow diameter section that includes the distal end of the rod.

The reduction in the diameter between the handling section 116 and the distal end 110 increases the flexibility. The flexibility reduces the force applied to the internal surface of the body cavity (e.g. nostril, mouth, rectum or other orifice) from where the sample is taken.

In other embodiments, the rod 102 has a consistent diameter along its length, or any other number of reductions in diameter.

The rod 102 also includes a weakened section 126 spaced from the tip 104. The weakened section 126 is presently in the form of a circumferential, V-shaped notch in the handling section 116. In other embodiments, the weakened section may be formed from a more brittle material than the rest of the rod.

After the tip 104 has been placed into VTM—e.g. in a tube or other receptacle—the weakened section 126 enables the rod 102 to be readily snapped so that the tip 104 remains in the VTM. To that end the weakened section 126 is spaced from the distal region 112 (more particularly, the distal tip of tip 104) by a distance just less than the internal length of the receptacle—presently, the weakened section 126 is in the handling section 116 but it may instead be located between the handling section 116 and the distal end 110. Accordingly, if the receptacle is jolted or tilted while being moved, the tip 104 will remain in the VTM.

The rod 102 facilitates control of the tip 104 during use—e.g. by a physician or nurse. The tip 104 is moved using the rod 102 to which it is attached, and is shaped for insertion into, and collection of the specimen from, the body cavity of the subject. With reference to FIG. 2, the tip 104 comprises a plurality of curved blades (some of which have been identified by reference 114) that define an internal reservoir 122 for receiving the specimen. During use, the tip 104 is rotated clockwise or counter-clockwise against the internal wall of the cavity of the subject to extract the sample. In the present embodiment, it is desirable that the tip 104 be rotated in a direction to cooperate with the direction of curvature of the blades 114—presently, direction X which is the same as the direction of rotation of the blades 114 from the proximal region 108 to the distal region 112.

Each blade 114 is continuous along the length L of the tip 104—i.e. the blades 114 each extend from the proximal region 108 to the distal region 112 and, in particular, from the proximal end 124 to the distal end 128. This ensure a sample can the taken into the reservoir over a large proportion of the length of the tip 104.

The blades 114 are equidistantly, circumferentially spaced around the tip 104. Thus, the gap 130 between neighbouring blades 114 is consistent while the tip 104 is not in use. While the tip 104 is in use, there may be some flexion of the shape of the gap 130.

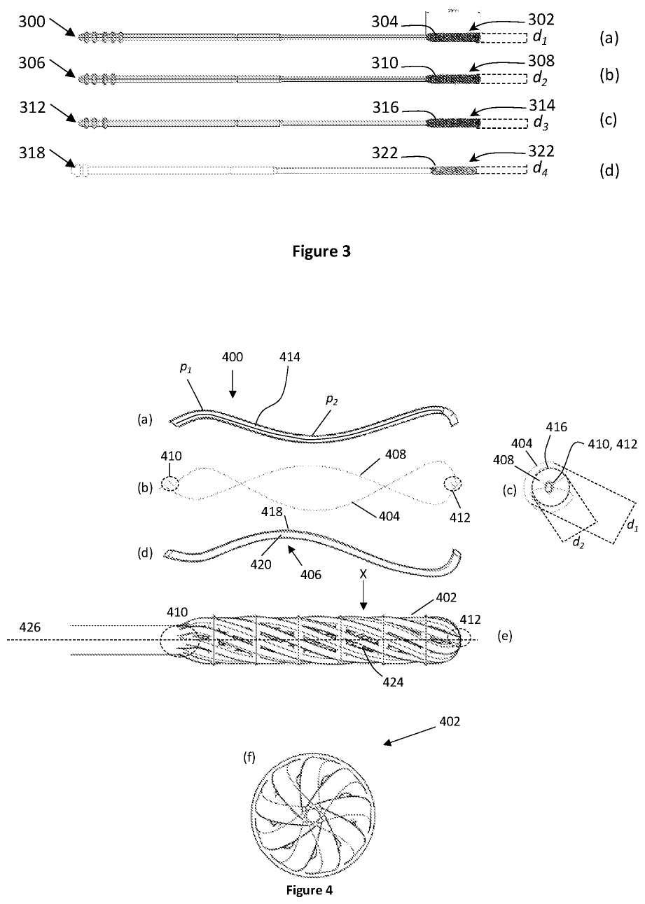

The swab 100 includes eight blades 114. However, the tip 104 may be formed from any desired number of blades. Moreover, the number of blades may depend on the shape or diameter of the tip 104, the viscosity of the sample being collected and other factors. FIG. 3, shows various embodiments of swabs, comprising between five and nine curved blades. FIG. 3a is a swab 300, including a 20 mm long tip 302 having five curved (presently helical) blades 304 and a diameter d₁ of 3.4 mm. FIG. 3b is a swab 306, including a 20 mm long tip 308 having seven curved (presently helical) blades 310 and a diameter d₂ of 3.4 mm. FIG. 3c is a swab 312, including a 20 mm long tip 314 having nine curved (presently helical) blades 316 and a diameter d₃ of 3.4 mm. FIG. 3d is a swab 318, including a 15 mm long tip 320 having seven curved (presently helical) blades 322 and a diameter d₂ of 3.0 mm.

Each blade 114 extends along a coterminous length of the tip 104, presently the entirety of length L. In other embodiments, the coterminous length may be less than L where, for example, a solid proximal end or distal end is provided to enhance strength or make it possible to include a greater number of narrower blades than may be supportable (i.e. functionally useful) without such a solid end. In addition, each of the blades 114 curves in a common direction, presently counter-clockwise when moving from the proximal region 108 to the distal region 112. This allows the blade 114 to have the same diameter—i.e. the full diameter of the tip 104.

The blades 114 may have a flat surface, or be textured to increase the hydrophilic surface area in contact with the internal wall of the body cavity, or to scrape more sample for each rotation of tip 104.

A square shape, helix design with a reservoir concept is applied to the swab 100. Each curved blade 114 is a helical blade. Thus, each blade 114 forms a helical shape extending along the tip 104. All blades 114 disclosed herein may have any required or desired number of turns of the helix—e.g. between one and two turns along tip 104. The present helix curve is designed to be 1.5 turns from start (proximal tip 124) to end (distal tip 128) resulting in a significant amount of surface manipulation (sculpting, flexing and weaving) while retaining sufficient structural integrity for withstanding pushing and turning impact forces.

FIG. 4a shows a single blade 400. Along its trajectory along the tip 402 it passes along a helix stroke path 404 shown in FIG. 4b. While the same helix stroke path 404 will apply to blades 114, FIG. 4 shows the present embodiment includes inner blades 406 (FIG. 4d) and outer blades 400 (FIG. 4a). The inner curved blades 406 are oppositely curved—i.e. where the outer blades 400 curve in one direction, either clockwise or counter-clockwise, the oppositely curved blades 406 will curve in the opposite direction, either counter-clockwise or clockwise respectively—and follow trajectory or helix stroke path 408. The stroke paths 404, 408 converge in both the proximal region 410 and distal region 412. This can also be seen in FIG. 4c, which more clearly shows the stroke path 408 of the oppositely curved blades 406, having a smaller diameter d₂, when compared with the stroke path 404 of the curved blades 400, having a larger diameter d₁ being the same as the diameter of the tip 402.

The blade 400 has a flat diamond cross-section, though square, diamond and other cross-sections may be used. Notably, the faces (one of which is referenced by numeral 414) of blade 400 shown in FIG. 4a face in the same direction along the length of the tip 402. This means that each face 414 will be an outer face at some point p₁ along the helix (i.e. in a position to contact the internal wall of the body cavity) and an inner face at some other point p₂ along the helix (e.g. defining part of the reservoir 122 of swab 100). As a result, as the tip 402 is rotated, sample is scraped from the cavity wall and, as it collects between the blades, sample scraped off during further rotation of the tip 402 will push previously scraped sample along the face 414 and thus into the reservoir. The sample will be retained in the reservoir since, as the relevant face 414 again becomes the surface in contact with the cavity wall, it will be blocked from travelling with the relevant face by freshly scraped sample.

The tip 402 therefore comprises a plurality of oppositely curved blades 406 (presently of helical shape), the oppositely curved blades 406 curving in a direction opposite to the common direction of the curved blades 400. Moreover, the oppositely curved blades 414 are offset inwardly of the curved blades 400. The present helix design creates eight blades 400 on the outside and four reverse blades (i.e. oppositely curved) 414 offset inwards reverse blades to perform the sculpting of cells (i.e. harvesting cells during rotation of the swab in the e.g. nasal cavity—scratching the nasal surface to gather cells within the tip) as well as deposit the cells into the reservoir 416 in the center of the tip.

The oppositely curved blades 414 also support the curved blades 400. This increases the resistance of the tip to plastic deformation—e.g. from squeezing by hand during removal of the swab from a packet. The oppositely curved blades 414 have an outer side (which may also refer to an edge) 418 for contacting the curved blades 400, thereby supporting the curved blades 400, and an inner side (which may also refer to an edge) 420. The internal reservoir 416 is defined between the inner sides 420 of the oppositely curved blades 414. In the embodiment of FIG. 1, that reservoir 122 is instead defined by the internal side or edge of the curved blades 114.

The structure of tip 402 further includes a plurality of perpendicular supports 422 for supporting or reinforcing the blades 404, 408 between opposite ends or region 410, 412 of the tip 402. These supports 422 help to resist plastic deformation from compressive forces—e.g. when a nurse presses on the tip during removal from the packaging. There may be no such supports 422, one such support 422 or more than one support 422 as required. The tip 402 is thus designed to resist laterally applied forces (in a direction along the trajectory marked X, normal to the axis 106) that would otherwise cause plastic deformation (e.g. micro-cracks), and potentially breakage, of the tip 402. The perpendicular support are presently perpendicular triangular ring (i.e. annular) blades running along the tip 402 at spaced intervals. The tip 402 is 20 mm long, including 3 mm at the proximal region in which the blades merge together into the rod such that the blades are 17 mm long, the diameter is 3 mm and the triangular ring supports are spaced at intervals of 2.6 mm centre-to-centre.

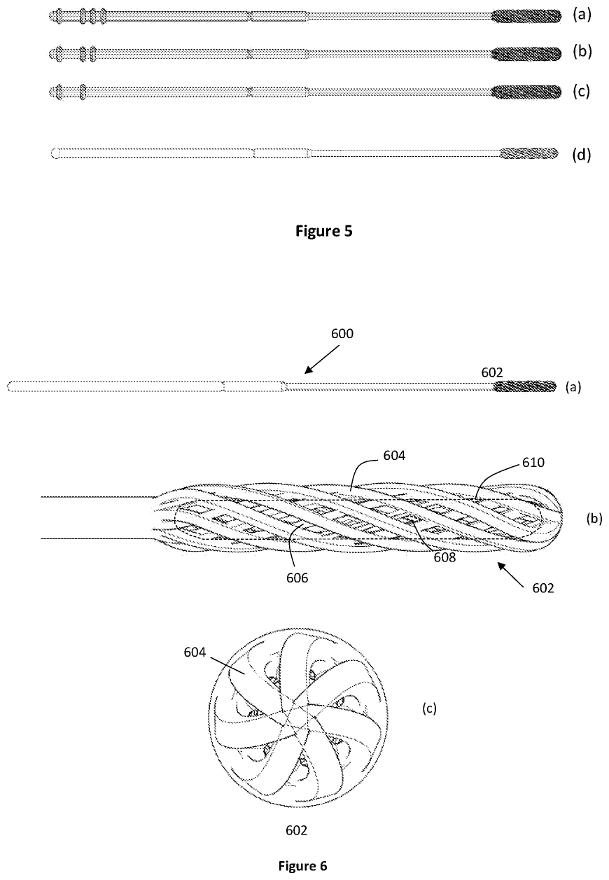

The tip 402 is further reflected in FIG. 4f, which shows the swab tip 402 with eight outer helixes (blades 400), four inner helixes (blades 406) and six perpendicular ring supports in end view (FIG. 5b).

In some embodiments, the curved blades (presently each a helix) were found to be most effective when continuous and connected from the distal region or distal end (or end first inserted into the body cavity—e.g. nostril) to the proximal region or proximal end (base—end of the tip last inserted into the body cavity) of the tip. The tip then rotates along a longitudinal axis—i.e. along the length or axis of the rod, through the proximal and distal ends of the tip—to maximise scraping action and structural strength. The tip mimics a drilling action (ability to scratch the surface for better cell/sample harvest) whilst the internal helix (oppositely curved blades) runs in the opposite direction and is offset inwards (offset by 0.5 mm inwards)—i.e. it is not on the same plane of the outer helix blade (curved blades)—such that it will not interfere with the scrapping effectiveness/action (produced by the outer helical blades) of the swab when it is rotated within the patient's nose.

The helixes (outer and inner sets of blades) weave together to provide good structural strength as well as adequate flexibility. They also form small diamond shape small pockets (i.e. diamond-shaped apertures) that are longer in the longitudinal direction—i.e. from direction Up (distal) Down (proximal) along the longitudinal axis of the swab—to allow better capture of samples when twisting in the opposite direction as shown in FIG. 2. The openings when untouched (no force or twisting added) will help with better liquid/sample retention in the reservoir within the swab.

FIG. 5 shows that various dimension adjustments and blade number adjustments can be made, while remaining within the scope of the present teachings. The effective versions of swab designs explores improved sculpting features (adding surface textures) by creating surface ridges at regular intervals whilst increasing surface area for sample capture. The effective range for single helix swabs was found to be from five to nine helixes (five helixes=more flexible and comfortable with wider gap between helixes, reduced sample retention or nine helix=more rigid and durable, with tighter gap for better sample retention) revolving around the centre of the swab stick or rod. Other numbers of helixes (blades) may also be used in some applications.

Outer diameter of the tip was also adjusted between 3 mm and 3.4 mm to optimize sample capture within the hollow internal reservoir. Both single or double helix structures were effective in certain configurations (e.g. dimensions and blade numbers) whilst being very good or ideal for absorption and retention during use and release when immersed in the viral transport medium.

For ease of reference, different indices—presently in the form of circumferential protrusions—are provided at the proximal end of each rod. These indices may be unique to each set of tip characteristics—i.e. variation in functional requirements such as variation in absorption, release, structural strength, tissue type (for different cavity types), and so on.

FIG. 5a provides a tip having nine outer helixes and five inner helixes, with 20 mm tip length (including the proximal region mentioned above) and 3.4 mm tip diameter. FIG. 5b provides a tip having nine outer helixes and seven inner helixes, with 20 mm tip length (including the proximal region mentioned above) and 3.4 mm tip diameter. FIG. 5c provides a tip having nine outer helixes and nine inner helixes, with 20 mm tip length (including the proximal region mentioned above) and 3.4 mm tip diameter. In contrast, FIG. 5d provides nine outer helixes and no inner helixes, in a 3.4 mm diameter tip.

An alternative embodiment is shown in FIG. 6, being an embodiment without perpendicular ring reinforcements. The swab 600 shown in FIG. 6a includes a tip 602 having nine outer helix square blades (curved blades 604—see FIG. 6b) that are interwoven with four reverse helix blades (oppositely curved blades 606) for effective absorption, retention and release of a sample. The tip 602 is shown in side view in FIG. 6b and end view in FIG. 6c. The reservoir space can be adjusted by adjusting the diameter of the tip—e.g. from 3 mm to 3.4 mm outer diameter for tested patient comfort and length of tip ranges from 17 mm to 20 mm. Tip 602 may have the same dimensions as tip 400, though without the supports.

The curved blades 604 and oppositely curved blades 606 together define diamond-shaped apertures (one of which is identified by reference numeral 424 in FIG. 4e and another identified by reference numeral 608 in FIG. 6b) through which the sample is conveyed into the reservoir 610 (indicated by broken line in FIG. 6b. The diamond-shaped apertures 424 are longer in a longitudinal direction (i.e. parallel to axis 426) of the swab than in a transverse direction (i.e. perpendicular to axis 426).

The reservoir 608 is generally cylindrical and formed by the inner edges, sides or surfaces of the innermost group of blades—i.e. the blades 114 of the embodiment of FIG. 1 and blades 406 of the embodiment of FIG. 4—where the outer edges, sides or surfaces of the outermost blades contact the internal wall of the body cavity. The reservoir 608 performs the retention of cells and mucus using capillary effect through the gaps 424, 610 created between the structural weaves (curved blades and oppositely curved blades) that allows for absorption and retention as well as the releasing of cells and mucus when submerged in the transport media.

This capillary effect is aided by the curved blades having some lateral flexibility. In use, portions of the blades will bend somewhat, against the direction of rotation, on coming into contact with the internal wall of the body cavity. Similarly, once that portion is no longer in contact with the internal wall, it will move back to its original (i.e. rest) position. This causes a small amount of relative movement between neighbouring blades as indicated by arrows Y in FIG. 2. That movement can assist in squeezing the scraped sample into the reservoir or between the oppositely curved blades. Thus, the curved blades are spaced (i.e. from each other) to transfer the specimen from the cavity to the reservoir under capillary action, and to assist that capillary. The blades have sufficient rigidity to scrape a sample from the internal wall of the cavity, and are sufficiently flexible to vary (i.e. by flexing) a spacing between neighbouring ones of said blades, during rotation of the swab in the cavity, to drive the sample into the reservoir.

The material properties of the swabs disclosed herein enable them to operate under the same conditions as those required in a nasopharyngeal swabbing process. As a result, a single 3-dimensional printing process can be used for production, without additional processes for the absorption of material on the tip—e.g. by flocking.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1.-23. (canceled)

24. A tip for a swab for collecting a specimen from a subject, the tip being shaped for insertion into, and collection of the specimen from, a body cavity of the subject, and comprising a plurality of helical curved blades defining an internal reservoir for receiving the specimen, the tip being attached or attachable to a rod.

25. The tip of claim 24, wherein the tip has a length and each blade is continuous along the length.

26. The tip of claim 24, wherein each said helical curved blade comprises between one and two helical turns.

27. The tip of claim 26, wherein each curved blade comprises about 1.5 helical turns.

28. The tip of claim 24, wherein the helical curved blades are equidistantly, circumferentially spaced around the tip.

29. The tip of claim 24, wherein the helical curved blades have an outer side for contacting an internal wall of the cavity, and an inner side, the internal reservoir being defined between the inner sides of the curved blades.

30. The tip of claim 29, wherein the helical curved blades are spaced to transfer the specimen from the cavity to the reservoir under capillary action.

31. The tip of claim 24, wherein each said helical curved blade extends along a coterminous length of the tip.

32. The tip of claim 24, wherein the helical curved blades curve in a common direction.

33. The tip of claim 32, wherein the tip further comprising a plurality of oppositely curved blades, the oppositely curved blades curving in a direction opposite to the common direction, the oppositely curved blades being offset inwardly of the curved blades, wherein each said oppositely curved blade is a helical blade.

34. The tip of claim 33, wherein the oppositely curved blades have an outer side for contacting the curved blades, and an inner side, the internal reservoir being defined between the inner sides of the oppositely curved blades.

35. The tip of claim 24, further comprising one or more perpendicular supports for supporting the helical curved blades between opposite ends of the tip.

36. The tip of claim 24, wherein each said helical curved blade comprises a square cross-section.

37. The tip of claim 24, wherein a surface of each one of the helical curved blades is textured.

38. The tip of claim 24, wherein the helical curved blades each form a helix and curve in a first direction, the tip further comprising a plurality of oppositely curved blades, the oppositely curved blades curving in a second direction opposite the first direction, the oppositely curved blades being offset inwardly of the curved blades, wherein the curved blades and oppositely curved blades define diamond-shaped apertures through which the sample is conveyed into the internal reservoir.

39. The tip of claim 38, wherein the diamond-shaped apertures are longer in a longitudinal direction of the swab than in a transverse direction.

40. The tip of claim 24, wherein the helical curved blades have sufficient rigidity to scrape a sample from an internal wall of the cavity, and are sufficiently flexible to vary a spacing between neighbouring ones of said helical curved blades, during rotation of the swab in the cavity, to drive the sample into the reservoir.

41. A swab comprising:
a tip according to claim 24; and
a rod having a distal end, the tip being located at the distal end.

42. The swab of claim 41, wherein the rod is integrally formed with the tip.

43. The swab of claim 41, wherein the rod is attached to the tip.
